(19) United States
(12) Patent Application Publication
(10) Pub. No.: US 2005/0048584 A1
(43) Pub. Date: Mar. 3, 2005

(54) Method for detecting alzheimer's disease and differentiating alzheimer's disease from other demential diseases associated peptides and use thereof

(76) Inventors: Norbert Lamping, Hannover (DE); Hans-Dieter Zucht, Hannover (DE); Harmut Selle, Hannover (DE); Michael Jurgens, Hannover (DE); Gabriele Heine, Hannover (DE); Rudiger Hess, Hannover (DE)

Correspondence Address: WHITHAM, CURTIS & CHRISTOFFERSON, P.C., 11491 SUNSET HILLS ROAD, SUITE 340, RESTON, VA, 20190, US

(21) Appl. No.: 10/497,073

(22) PCT Filed: Nov. 27, 2002
(86) PCT No.: PCT/DE02/04360

(30) Foreign Application Priority Data
Nov. 28, 2001 (DE) 101 58 180.7

(51) Int. Cl. C12Q 1/68; G01N 33/53; G01N 33/567

(52) U.S. Cl. 435/7.2; 435/6

(57) ABSTRACT

The invention relates to defined peptide and the quantiative determination thereof in the body fluids of patients suffering from Alzheimer's disease in relation to the concentration thereof in a control group of patients who are healthy or suffer from other dementia diseases. The inventive peptides originate from the C3f fragment of the complement C3 protein precursor with the corresponding gene and are modified in a specific manner and are optionally post-translationally or chemically modified. Modification occurs with respect to the concentrations of said peptides in patients in a manner which is specific to each peptide when compared with the control group. A specific and significant modification of the concentration of said peptides in relation to the concentration thereof in healthy persons indicates a differentiation with respect to Alzheimer's disease. The invention also relates to the use of said peptides as a progress control, and in the development of diagnostic and therapeutic agents.

The invention relates to a method for detecting Alzheimer's disease, in particular a method which makes it possible to differentiate Alzheimer's disease from other dementia diseases. For this purpose, the concentration of particular peptides in body fluids or other samples from the patient is measured. The invention further relates to peptides, antibodies, nucleic acids etc. which have been found for determining the presence and/or the grade of the disease, and to uses thereof for diagnosis and therapy.

Dementia diseases represent an increasing problem because of the demographic shift toward and ever longer life expectancy since they occur to an increasing extent as age advances. Dementia diseases are in most 20 cases incurable and make long-term care of the patients necessary.

About half of these patients receive in-patient care. More than 60 dementia diseases are known, including diseases associated with manifestations of dementia. Alzheimer's disease is the commonest disease in the group of dementia diseases [1]. The diagnosis and therapy of Alzheimer's disease is therefore of particularly great importance. Alzheimer's disease is a neurodegenerative disease which is distinguished by the following symptoms: decline in intellectual abilities, short-term and long-term memory impairments, confusion and diminished ability to look after oneself. The deterioration in intellectual capacity occurs in addition to the age-related deterioration in intellectual abilities [1].

Diagnosis of Alzheimer's disease is difficult because it, just like other dementia diseases, has an insidious onset and is associated with a slowly progressive destruction of nerve cells in the brain and, associated therewith, diminished mental capacity. Various other dementia diseases, such as, for example, vascular dementia, display very similar symptoms, which makes it very difficult to differentiate diagnostically between Alzheimer's disease and other dementia diseases.

A widely used method for diagnosing dementia diseases is determination of the MMSE score (mini-mental status examination, MMSE) [2]. However, this test does not allow different dementia diseases to be reliably differentiated. Clinically measurable laboratory parameters which can be used are determination of the concentration of tau protein and of the beta-amyloid isoform which is 42 amino acids in size, there being an increase in tau and a decrease in beta-amyloid in the cerebrospinal fluid of patients with Alzheimer's disease. Although both markers display high sensitivity, they display only low specificity. This greatly restricts their diagnostic value, so that it can be said that at present no diagnostic marker for reliable diagnosis of Alzheimer's disease is available [3].

Since at present no reliable diagnosis of Alzheimer's disease is possible, nor is effective therapy, the provision of a reliable, clinically measurable parameter for detecting Alzheimer's disease and for differentiating this neurodegenerative disease from other dementia diseases represents an important medical advance. The present invention additionally has great importance for the development of therapies for the treatment of Alzheimer's disease because a reliable diagnosis of the disease to be treated is a precondition for developing therapies. This is particularly important when different diseases with similar symptoms exist, but these diseases presumably have different causes and therefore require different therapies. Alzheimer's disease cannot at present be reliably differentiated from other dementia diseases [3].

The invention is based on the object of avoiding the prior art disadvantages in the diagnosis of Alzheimer's and of providing a method which can be used early and reliably for detecting and for differentiating Alzheimer's disease from other dementia diseases.

It has surprisingly been found that only in samples of body fluids from patients suffering from Alzheimer's disease, especially in the cerebrospinal fluid, does the concentration of particular peptides differ from a reference value and makes it possible to detect Alzheimer's disease and to differentiate patients with Alzheimer's disease from patients with other dementia diseases and healthy people. Changes in the concentration of these peptides relative to the reference value, which can be determined for example on the basis of control samples, indicate the presence of Alzheimer's disease and are therefore suitable for the selective detection of this disease with high sensitivity and specificity.

The peptides are fragments of the precursor molecule complement C3, in particular a defined peptide which is 17 amino acids long and is derived from complement C3 and which is referred to as C3f [4]. It was additionally possible to detect a further C3f peptide in which the C-terminal amino acid arginine is eliminated (C3f-des-Arg) in the cerebrospinal fluid from patients with Alzheimer's disease in concentrations which differs from the concentrations of the respective peptide in people with non-Alzheimer dementia or healthy people.

We have been able to detect and identify further novel previously unknown variants of the C3f peptide in human hemofiltrate and plasma, and they can therefore also be used for diagnostic purposes in patients with Alzheimer's disease.

Not only the term C3f but also the term C3F occurs in the literature, and C3F is not identical to C3f. On the contrary, C3F stands for “C3-fast”, a complement C3 polymorphism which has the effect that the complete precursor molecule of this C3 variant migrates more rapidly in the electric field of the gel electrophoresis.

Hereinafter, the C3f peptide and the C3f peptide variants derived therefrom are referred to as “Alzheimer's disease complement C3 peptides” (ADC3 peptides). The sequence of C3f is identical to the sequence Seq. ID 1 from the sequence listing, and to the sequence of ADC3-1. ADC3 peptides comprise at least 8 and at most 17 amino acids, which are identical to the amino acids at the corresponding sequence positions in the sequence of C3f corresponding to Seq. ID 1. In addition, ADC3 peptides may comprise two point-mutated, two deleted or two additional internally inserted amino acids, and N-terminal and/or C-terminal extensions corresponding to Seq. ID 1. However, in these cases they must retain at least 8 amino acids from the sequence from Seq. ID 1 at the appropriate sequence positions. In addition, C3f peptides and C3f peptide fragments derived from naturally occurring complement C3 polymorphisms and from naturally occurring C3 mutants are also referred to as ADC3 peptides.

To achieve the object, the invention encompasses a method for detecting Alzheimer's disease by determining the concentration of at least one marker peptide in a biological sample from a patient that at least one ADC3 peptide is used as marker peptide, a concentration increase or concentration reduction, which is specific for the respective marker peptide, is found for the marker peptide in the sample, a marker peptide concentration change in the aforementioned manner is regarded as positive detection result for Alzheimer's disease.

In this connection, it is possible in principle for each ADC3 peptide either to undergo only an increase in the peptide concentration in patients with Alzheimer's disease, or it is possible in principle for an ADC3 peptide to undergo only a reduction in the peptide concentration in patients with Alzheimer's disease. For a defined ADC3 peptide it is not possible for the ADC3 peptide concentration simultaneously to be increased in one individual patient with Alzheimer's disease and to be reduced in another patient with Alzheimer's disease. As with virtually all medical diagnoses of diseases, however, it is possible for a diagnosis to be wrong in a few individual cases because the concentration of the ADC3 peptides does not differ with hundred percent probability in each individual patient with Alzheimer's disease from the concentration of the ADC3 peptides in control samples.

Peptides which can be regarded as fragments of the complement C3f sequence, or the C3f peptide itself, are referred to as ADC3 peptides for the purposes of this invention. They include homologous peptides and peptide fragments derived from the complement C3f fragment. They include derivatives of naturally occurring alleles of these peptides and homologous mutants, in particular point-mutated mutants with preferably not more than two amino acids differing from complement C3. Preferred markers according to the invention are indicated in the sequence listing and are numbered from ADC3-1 to ADC3-16, corresponding to Seq. ID 1 to 16, where ADC3-1 corresponds to Seq. ID 1, ADC₃₋₂ to Seq. ID 2, ADC3-3 to Seq. ID 3 etc.

Homologous sequences mentioned in the present application mean sequences having at least 70 percent homology. The homology between sequences can be determined using computer programs such as, for example, the GCG program package (Genetics Computer Group, University of Wisconsin, Madison, Wis., USA), including GAP [5], BLASTP, BLASTN, FASTA [6] or the well-known Smith Waterman algorithm for determining homologies. Preferred parameters for the amino acid sequence comparison comprise the algorithm of Needleman and Wunsch [7], the comparison matrix BLOSUM 62 [8], a gap penalty of 12, a gap length penalty of 4 and a threshold of similarity of 0. The GAP program is also suitable for use with the aforementioned parameters. The aforementioned parameters are the default parameters for amino acid sequence comparisons, where gaps at the ends do not reduce the level of homology. When sequences are very short compared with the reference sequence, it may additionally be necessary to increase the expectation value as far as 100000 and, where appropriate, to reduce the word size as far as 2. Further examples of algorithms, gap opening penalties, gap extension penalties, comparison matrices including those mentioned in the program handbook, Wisconsin package, version 9, September 1997, can be used. The selection will depend on the comparison to be carried out and additionally on whether the comparison is carried out between sequence pairs, in which case GAP or Best Fit are preferred, or between a sequence and a comprehensive sequence database, in which case FASTA or BLAST are preferred. An agreement of, for example, 70% found using the abovementioned algorithm is referred to as 70% homology. Homologous sequences in the present application mean in principle all sequences having at least 70% agreement, where the agreement is calculated in accordance with the aforementioned description. This refers both to amino acid sequences and to nucleic acid sequences.

The marker peptides may be in the form of post-translational modifications and/or in enzymatically and/or chemically modified form, preferably as peptide oxides, and be detected in this form. A chemically or post-translationally modified complement C3 peptide or ADC3 peptide may consist both of D- and of L-amino acids, and of combinations of D- and L-amino acids, and may either occur naturally, be produced recombinantly or enzymatically or be synthesized chemically. These peptides may additionally comprise unusual amino acids, i.e. amino acids which do not belong to the 20 standard amino acids. Numerous examples of unusual amino acids and pos-translational modifications such as, for example, phosphorus and sulfate groups, glycosilations, amidations, deamidations, pyroglutamic acid etc. are described in the literature and in databases [9].

It is additionally possible for the purposes of the invention for one or more amino acids or the entire peptide to be replaced by structures consisting of peptidomimetics. The term peptidomimetics is used in the present application in the form of the widest possible definition. A peptidomimetic is a substance which comprises non-peptide structural elements and is able to imitate or antagonize the biological effect of the natural parent molecule. Numerous studies which deal in detail with possibilities of using peptidomimetics as substitute for conventional peptide structures are known in the prior art.

Nucleic acids are regarded as being DNA, RNA and DNA-RNA hybrid molecules both of natural origin and prepared synthetically, enzymatically or recombinantly. Also included are nucleic acids which comprise modified nucleotides with altered in vivo stability, such as, for example, phosphorothioates. Such stabilized nucleic acids are already used in the application of ribozyme, antisense RNAi (“RNA-mediated interference”) and triplex nucleic acid techniques.

The method of the invention is a method in which there is measurement of specific biomarkers whose concentration is changed in neurodegenerative diseases, especially in Alzheimer's disease, and which indicate the disease even at a very early stage and indicate an increased probability of a disease risk at an early time. This is important in order to provide a reliable clinical marker for diagnosing these diseases.

It is possible and preferable for the concentration of the ADC3 peptides in the sample, but also the characteristic pattern of DC currence of a plurality of particular ADC3 peptides, to be correlated with the severity of the disease. These novel markers therefore make it possible to develop and monitor therapies for the treatment of Alzheimer's disease, because the course and any successful cure resulting from a therapy or a diminished progression of the disease can be established. Effective therapy of Alzheimer's disease is not possible at present, underlining the urgency for the provision of a reliable detection method for Alzheimer's disease Alzheimer's disease, because reliable detection of the disease is a precondition for the development of a therapy.

Detection of ADC3 peptides additionally makes it possible in the framework of clinical studies to develop novel therapies for the treatment of Alzheimer's disease with high specificity to select only those patients suffering from Alzheimer's disease and not from other, similar diseases. This is important for obtaining valid study results, because patients incorrectly diagnosed as patients with Alzheimer's disease have a negative influence on the quality of the results of a study on Alzheimer's disease therapy.

Complement C3 Biology

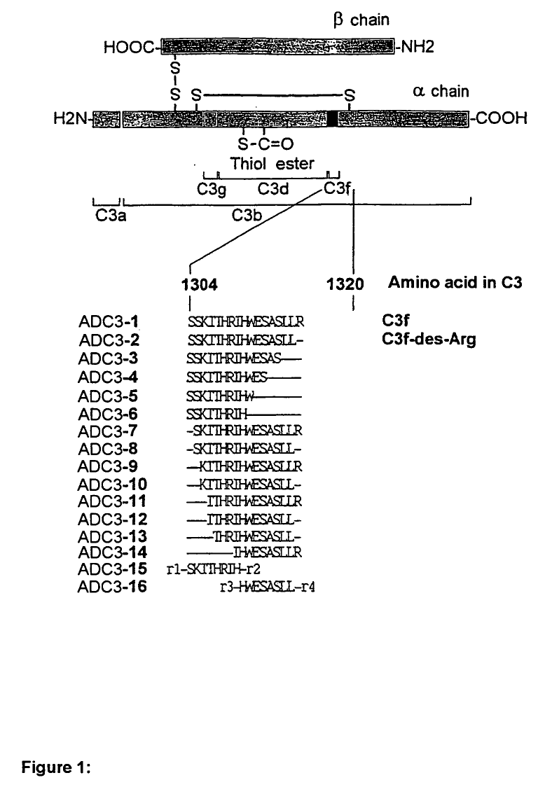

The complement system plays a central role in specific and nonspecific immune defences and consists of more than 30 proteins, of which some are proteases and others, in turn, are substrates of these proteases. The mode of functioning of the complement system is based on a type of chain reaction at the start of which a substance, e.g. a bacterial cell membrane, activates a component of the complement system, this complement factor in turn activates the next complement factor etc. The complement activation may also lead to lysis of the host's own healthy cells which are by chance in the vicinity of the complement activation site. This process is also called the bystander effect, and it may result in wide-ranging tissue damage. Some peptides released during the complement cascade by proteolytic cleavage of individual complement proteins, especially the anaphylatoxins C3a and C5a, stimulate immune cells in a variety of ways and thus lead to a local inflammatory reaction [10]. Anaphylatoxin C3a has, for example, cytotoxic, vasodilating and cell-stimulatory properties. Besides anaphylatoxin C3a, a number of other complement C3 cleavage products are known, such as, for example, C3b, C3c, C3d, C3g, C3e and C3f (FIG. 1). A biological function is known for some of these C3 peptides, but not for all.

Factor I eliminates from C3b initially a small peptide with a length of 17 amino acids, C3f, and subsequently several other defined C3 peptides, including C3dg. C3dg is then cleaved further to C3d and C3g. C3b is inactivated in this way [11]. The C3d peptide is the C3 peptide in which the thiol ester group is present in the intact C3 molecule, and which therefore occurs covalently linked to complement-activating substances. Scientific investigations show that C3f in some cases mediates biological effects similar to C3a. C3f and C3a lead to contraction of smooth muscle and increased vascular permeability [11]. In addition, the C-terminal amino acid in C3a and in C3f is an arginine, and elimination of this amino acid changes the biological activity both of C3a and of C3f. The resulting products are called respectively C3a-des-Arg and C3f-des-Arg. It is presumed that C3f and C3a use, at least in part, the same receptors, which might explain their similar biological effects [11].

C3 is synthesized mainly by the liver, but also locally by monocytic cells and macrophages. During bacterial meningitides, i.e. in bacterial infections in the brain, C3 concentrations in the cerebrospinal fluid are increased about 20-fold. There is likewise an increase in the C3 concentration in the CSF in Alzheimer's disease, but only by a factor of two, as the C3 concentration in the cerebrospinal fluid remains unchanged in other inflammatory reactions in the brain such as, for example, an aseptic meningitis [12]. Complement proteins and their receptors are also synthesized inter alia by microglia, astrocytes and by neurons. The amount of mRNA and protein of the complement proteins C1 to C9 is increased in the brain of patients with Alzheimer's disease relative to the brain of healthy people. The concentration of C3 and C4 is changed least relative to healthy people, with a 2-fold increase in the brain and unchanged concentrations in the liver. Various other complement proteins are increased considerably more in the brain of patients with Alzheimer's disease relative to healthy people: C1q mRNA is increased 23-fold, C1r 5-fold, C7 6-fold and C9 is increased 22-fold [12]. It is therefore surprising and unexpected that fragments of the complement C3 protein and not fragments of other complement proteins are suitable as markers of Alzheimer's disease.

Deposits present in the form of plaques and tangles in the brain of patients with Alzheimer's disease consist mainly of beta-amyloid and tau protein. Besides these main components, however, a large number of other proteins has been identified immunohistologically in these deposits. These proteins include, inter alia, 1-antichymotrypsin, synaptophysin, cystatin C, heme oxigenase, various apolipoproteins such as Apo E4, Apo J and Apo A1, IL-6, the AMY antigen, prion protein, beta-spectrin, alpha-2-macroglobulin, lactoferrin and various complement proteins such as C1q, C3 and C4d. In addition to proteins, these deposits also comprise sugar structures such as, for example, heparan sulfate. These deposits thus represent complex structures of proteins and other substances. It was discovered in 1982 by Eikelenboom et al. that deposits in the brain of patients with Alzheimer's disease contain complement C3 inter alia, and that beta-amyloid presumably activates the complement cascade, and it was possible to show this experimentally in subsequent studies [13].

Preferably Embodiments of the Invention

We have detected the complement C3 peptide C3f and various partial sequences of C3f in various body fluids such as, for example, hemofiltrate, plasma or cerebrospinal fluid. The peptides ADC3-3 to ADC3-16 are C3f peptide fragments which have never to date been described in the literature and are therefore novel as substances. ADC3-1 and ADC3-2 are already known from the literature [4, 11]. However, one of the peptides ADC3-1 to ADC3-16 has to date been suggested to be connected with neurodegenerative diseases, and consequently their use as diagnostic markers for detecting Alzheimer's disease is novel. This is the case in particular because it is known that the precursor molecule complement C3 is processed to numerous known and accurately defined peptides, and it was therefore unexpected and could not have been predicted by the skilled worker that only the C3f peptide and previously unknown C3f peptide fragments from the complement C3 precursor molecule can be used for diagnosing Alzheimer's disease, whereas no other complement C3 peptides were identified as suitable markers.

| | | Mono- | |
|---|---|---|---|
| ADC3 | C3 | isotopic | |
| pep- | sequence | theo- | |
| tide | position | retical | |
| no. | (A.A.) | mass (Da) | Peptide sequence |
| 1 | 1304-1320 | 2020.0966 | SSKITHRIHWESASLLR |
| 2 | 1304-1319 | 1863.9955 | SSKITHRIHWESASLL |
| 3 | 1304-1317 | 1637.8274 | SSKITHRIHWESAS |
| 4 | 1304-1315 | 1479.7583 | SSKITHRIHWES |
| 5 | 1304-1313 | 1263.6836 | SSKITHRIHW |
| 6 | 1304-1312 | 1077.6043 | SSKITHRIH |
| 7 | 1304-1320 | 1933.0646 | SKITHRIHWESASLLR |
| 8 | 1305-1319 | 1776.9635 | SKITHRIHWESASLL |
| 9 | 1306-1320 | 1846.0326 | KITHRIHWESASLLR |
| 10 | 1306-1319 | 1689.9315 | KITHRIHWESASLL |
| 11 | 1307-1320 | 1717.9376 | ITHRIHWESASLLR |
| 12 | 1307-1319 | 1561.8365 | ITHRIHWESASLL |
| 13 | 1308-1319 | 1448.7524 | THRIHWESASLL |
| 14 | 1311-1320 | 1210.6459 | IHWESASLLR |
| 15 * | 1305_{+r1″} | ≧990.5723 | r1-SKITHRIH-r2 |
| | 1311₊ᵣ₂ | | |
| 16 | 1312_{+r3″} | ≧941.4607 | r3-HWESASLL-r4 |
| | 1319₊ᵣ₄ | | |
| * r1″ represents either the amino acid serine or no amino acid is present at this sequence position, r2″ represents a sequence which corresponds to the sequence or parts of the sequence of complement C3 for amino acid 1313 to 1320, and r2 can be between 0 and 8 amino acids long, starting from amino acid 1312 of the C3 precursor. Correspondingly, r3″ represents the C3 precursor | | | |
| # sequence for amino acid 1304 to 1311 or parts thereof, and r3 can be between 0 and 8 amino acids long, starting from C3 amino acid 1312. r4 corresponds to the amino acid arginine or, alternatively, no further amino acid is present at this position in the peptide sequence. | | | |

r1—represents either the amino acid serine or no amino acid is present at this sequence position, r2—represents a sequence which corresponds to the sequence or parts of the sequence of complement C3 for amino acid 1313 to 1320, and r2 can be between 0 and 8 amino acids long, starting from amino acid 1312 of the C3 precursor. Correspondingly, r3—represents the C3 precursor sequence for amino acid 1304 to 1311 or parts thereof, and r3 can be between 0 and 8 amino acids long, starting from C3 amino acid 1312. r4 corresponds to the amino acid arginine or, alternatively, no further amino acid is present at this position in the peptide sequence.

The ADC3 peptides of the invention can exist in post-translational or chemical modification forms, thus influencing inter alia their masses and thus the identification by mass spectrometry and also their elution behavior on chromatography, such as, for example, on reverse phase chromatography. In particular, the peptides may be in oxidized, phosphorylated, glycosilated, sulfated, amidated form etc. in the sample to be investigated. Naturally occurring C3f peptides and C3f peptide fragments derived from complement C3 polymorphisms or C3 mutants are likewise referred to as ADC3 peptides. The computer programs and algorithms used to determine that at least 8 amino acids agree with the C3f sequence (Seq. ID 1) are also used for determining the homology of sequences [5-8].

The peptides are also regarded as ADC3 peptides in particular when a maximum of 2 of their amino acids differ from the corresponding sequence of the C3 precursor molecule. It is permissible in this connection for there to be point mutations, deletions, insertions of amino acids and N-terminal and/or C-terminal extensions as long as the peptide sequence is between 8 and 17 amino acids long, at least 8 amino acids are conserved relative to the C3 precursor sequence, and a maximum of 2 amino acids differ from the C3f sequence.

For a positive detection of the disease, it is provided for the concentration of the identified ADC3 peptide(s) to be changed for each individual one of these peptides in a defined concentration which is either always specifically higher or always specifically lower for the particular peptide. The concentrations of the particular peptides can be used to determine the severity of the Alzheimer's disease, in particular as substitute or as supplement to carrying out a mini-mental status examination (MMSE).

Control samples which are possibly used may be a pooled sample from various controls. The Alzheimer's disease sample to be investigated may also be a pooled sample, and where there is a positive result individual investigations are carried out. A reference value for the particular individual ADC3 peptide can be determined from the results of the determination of individual ADC3 peptides in control samples. It is then possible in future measurements to differentiate patients suffering from Alzheimer's disease from patients not suffering from Alzheimer's disease to compare the determined measurement with this reference value in order to obtain a diagnosis.

The body fluid sample may preferably be (human) cerebrospinal fluid (CSF) or a sample of another biological material such as, for example, serum, plasma, urine, stool, tear fluid, synovial fluid, lymph, etc. This depends inter alia on the sensitivity of the chosen detection method (mass spectrometry, ELISA etc.). Cell or tissue samples may also be used where appropriate. It is therefore provided in a further embodiment of this invention for cell or tissue homogenates to be produced, for example from human tissue samples obtained during biopsies, or from blood cells, for preparation of the sample to be investigated. These tissues can be comminuted for example with manual homogenizers, with ultrasound homogenizers or with electrically operated homogenizers such as, for example, Ultraturrax, and subsequently be boiled in a manner known to a skilled worker in acidic aqueous solutions with, for example, 0.1 to 0.2 M acetic acid for 10 minutes. The extracts are subsequently subjected to the respective detection method, e.g. a mass spectrometric investigation. The samples can be prepared, for example where appropriate diluted or concentrated, and stored in the usual way.

Use of the ADC3 Peptides

The invention further encompasses the use of at least one of the ADC3 peptides of the invention for the diagnosis of neurodegenerative diseases, especially Alzheimer's disease, and the use of ADC3 peptides for obtaining antibodies or other agents which, because of their ADC3 peptide-specific binding properties, are suitable for developing diagnostic reagents for detecting this disease. The invention also encompasses the use of ADC3 peptides for obtaining phage particles which specifically bind these peptides, or which conversely present ADC3 peptides on their surface, and thus make it possible to identify binding partners of ADC3 peptides.

Detection Methods for ADC3 Peptides

Various methods can be used for detecting the ADC3 peptides within the framework of the invention. Methods suitable for this are all those which make it possible to detect ADC3 peptides specifically in a patient's sample. Suitable methods are, inter alia, physical methods such as, for example, mass spectrometry or liquid chromatography, molecular biology methods such as, for example, reverse transcriptase polymerase chain reaction (RT-PCR) or immunological detection techniques such as, for example, enzyme linked immunosorbent assays (ELISA).

Physical Detection Methods

One embodiment of the invention is the use of physical methods which are able to indicate the peptides of the invention qualitatively or quantitatively. These methods include, inter alia, mass spectrometry, liquid chromatography, thin-layer chromatography and NMR (nuclear magnetic resonance) spectroscopy etc. Quantitative measured results from a sample to be investigated, which were obtained for a group of patients suffering from neurodegenerative diseases, preferably Alzheimer's disease, are used for this. The probability of the presence of a disease and/or the severity of this disease can be inferred from these results.

In a preferred embodiment of this invention, the peptides in the sample are separated by chromatography before the identification, in particular preferably by reverse phase chromatography, with particular preference for separation of the peptides in the sample by high-resolution reverse phase high-performance liquid chromatography (RP-HPLC). A further embodiment of this invention is the carrying out of precipitation reactions to fractionate the sample using precipitants such as, for example, ammonium sulfate, polyethylene glycol, trichloroacetic acid, acetone, ethanol etc. The fractions obtained in this way are then subjected to the particular detection method, e.g. the mass spectrometric investigation. Liquid phase extraction is used in a further embodiment of the invention. For this purpose, the sample is mixed for example with a mixture of an organic solvent such as, for example, polyethylene glycol (PEG) and an aqueous salt solution. Owing to their physical properties, particular constituents of the sample then accumulate in the organic phase, and others in the aqueous phase, and can thus be separated from one another.

Reverse Phase Chromatography

A particularly preferred embodiment of this invention encompasses the use of reverse phase chromatography, in particular a C18 reverse phase chromatography column using mobile phases consisting of trifluoroacetic acid and acetonitrile, for separation of peptides in human cerebrospinal fluid. For example the fractions collected in each case each comprise 1/100 of the mobile phase volume used. The fractions obtained in this way are analyzed with the aid of a MALDI mass spectrometer (matrix-assisted laser desorption and ionization) using a matrix solution consisting of, for example, L(−) fucose and alpha-cyano-4-hydroxycinnamic acid dissolved in a mixture of acetonitrile, water, trifluoroacetic acid and acetone, and thus the presence of particular masses is established and the signal intensity quantified. These masses correspond to the masses of the peptides ADC3-1 to ADC3-16 of the invention.

Mass Spectrometry

In a preferred embodiment of the invention, ADC3 peptides can be identified with the aid of mass spectrometric determination, preferably a MALDI (matrix-assisted laser desorption and ionization) mass spectrometry. In this case, the mass spectrometric determination further preferably includes at least one of the following mass signals, in each case calculated on the basis of the theoretical monoisotopic mass of the corresponding peptide. It is possible for slight differences from the theoretical monoisotopic mass to show owing to the experimental error and the natural isotope distribution. In addition, in MALDI mass determinations a proton is added to the peptides owing to the method of measurement, whereby the mass increases by 1 dalton. The following masses correspond to the theoretical monoisotopic masses of the peptides identified by us; calculated with suitable software, in this case GPMAW 4.02. These theoretical monoisotopic masses may occur singly or in combination in a sample: ADC3-1=2020.1/ADC3-2=1863.9/ADC3-3=1637.8/ADC3-4=1479.7/ADC3-5=1263.6/ADC3-6=1077.6/ADC3-7=1933.0/ADC3-8=1776.9/ADC3-9=1846.0/ADC3-10=1689.9/ADC3-11=1717.9/ADC3-12=1561.8/ADC3-13=1448.7/ADC3-14=1210.6/ADC3-15≧990.5 and ADC3-16≧941.4 dalton. In addition, the experimentally determined masses of 2038 and/or 2054 may appear. These two experimentally determined masses relate to ADC3-1 respectively as mono- and as dioxidized peptide. The symbol≧(is greater than or equal to) is to be understood to mean that the relevant ADC3 peptides cannot have arbitrary larger masses but can have only the masses possible owing to the amino acids which are possibly additionally present at the ends of these peptides. The peptide can overall have a maximum length of 17 amino acids. Amino acids which may be additionally present at the ends of these peptides are not just any ones but only those which may be present at this sequence position owing to the sequence of the complement C3 precursor molecule.

Mass Spectrometric Determination of the Sequence of the ADC3 Peptides

For the further practical application of this embodiment, further confirmation of the result of detection is advisable and possible by establishing the identity of the peptides corresponding to the masses, taking account exclusively of peptide signals which may be derived from the complement C3f peptide. This confirmation takes place by identifying the peptide signals preferably using methods of mass spectrometry, e.g. MS/MS analysis [14].

Specific peptide fragments of the complement C3f peptide, and the C3f peptide itself (corresponding to ADC3-1), have been identified for the first time by the method of the invention, and their significance has been recognized. These C3f peptides and their derivatives are referred to herein as ADC3-1 to ADC3-16. Their sequences are indicated in the sequence listing under Seq. ID 1 to Seq. ID 16. The ADC3 peptides mentioned under Seq. ID 15 and 16 (ADC3-15 and ADC3-16) may comprise at the N and/or C terminus additional amino acids corresponding to the corresponding sequence of the complement C3f peptide (Seq. ID 1, ADC3-1). The invention also encompasses the ADC3 peptides prepared recombinantly or synthetically, and isolated from biological samples, in unmodified, chemically modified or post-translationally modified form. In this connection, two point mutations and other differences are possible as long as the ADC3 peptide has at least 8 amino acids which agree in their identity and their position within the peptide sequence with the complement C3f peptide.

Molecular Biology Detection Techniques

Finally, the invention also encompasses nucleic acids which correspond to complement C3 fragments, and especially those which correspond to the ADC3 peptides of the invention, the use thereof for the indirect determination and quantification of the relevant protein molecules. This also includes nucleic acids which represent, for example, noncoding sequences such as, for example, 5′- or 3′-untranslated regions of the mRNA, or nucleic acids which show a sequence agreement with the complement C3 nucleic acid sequence which is sufficient for specific hybridization experiments and which are therefore suitable for the indirect detection of relevant peptides, especially the ADC3 peptides.

One exemplary embodiment thereof encompasses the obtaining of tissue samples from patients and the subsequent determination of the concentration of an RNA transcript corresponding to the gene of complement C3 or corresponding to homologous genes. This entails comparison of quantitative measured results (intensities) from a sample to be investigated with the measurements obtained in a group of patients suffering from Alzheimer's disease Alzheimer's disease and a control group. Methods which can be used for the quantification are, for example, reverse transcriptase polymerease chain reaction (RT-PCR) or Northern blots in a manner known to the skilled worker. The probability of the presence of Alzheimer's disease and/or the severity thereof can be inferred from the results.

Immunological Detection Methods

In a further preferred embodiment of the invention, the ADC3 peptides can be identified using an immunological detection system, preferably an ELISA (enzyme linked immuno sorbent assay). This immunological detection picks up at least one ADC3 peptide. To increase the specificity, it is also possible and preferred to use the so-called sandwich ELISA in which the detection of the ADC3 peptide depends on the specificity of two antibodies which recognize different epitopes within the same molecule. However, it is also possible to use other immunological methods, e.g. direct or competitive immunological methods, to detect ADC3 peptides. Other ELISA-like detection techniques such as, for example, RIAs (radio immuno assay), ELI-Spot, luminescence chemoluminescence, electrochemoluminescence, fluorescence or bioluminescence immunoassay methods etc. are also suitable as immunological detection systems for ADC3 peptides. ADC3 peptides isolated from biological samples, recombinantly prepared or chemically synthesized can be used as standard for the quantification. Identification of the ADC3 peptide(s) is generally possible for example with the aid of an antibody directed to the peptide or peptide fragment, i.e. an antibody specific for the peptide. Further methods suitable for detecting peptides are, inter alia, Western blotting, immunoprecipitation, dot-blots, plasmon resonance spectrometry (BIACORE™), phage particles, PNAs (peptide nucleic acids), affinity matrices etc. Substances/molecules suitable as detection agents are generally all those permitting the construction of a specific detection system.

In all these immunoassay methods, it is preferred for the binding partner which is specific for the ADC3 peptide to be immobilized on a suitable support. Supports which can be used are all known supports such as, for example, plastic tubes, microtiter plates, particles, microparticles, protein chips etc. The binding partner can be immobilized directly on this support or the solid phase by adsorption or covalent coupling. A further possibility is to immobilize the binding partner for the ADC3 peptide individually via one pair of a binding pair such as, for example, biotin/avidin, biotin/streptavidin, antigen/antibody etc. These methods are known to the skilled worker.

The invention further relates to a test kit for detecting Alzheimer's disease or a predisposition to Alzheimer's disease, which comprises at least one binding partner, preferably an antibody which is directed against an ADC3 peptide. This binding partner is present in immobilized form bound to a suitable solid phase or a support, or may be provided in a form which makes immobilization possible. For example, the binding partner may be biotinylated if the biotin/avidin interaction is to be utilized as indirect binding.

The binding partner may, however, likewise be present in labeled form, or in a form which makes labeling possible, in the test kit. Indirect labeling can be inserted via the interaction of a binding pair such as avidin/biotin, or streptavidin/biotin or digoxin/antidigoxin antibody. These methods are known to the skilled worker.

The test kit may additionally, besides the immobilized or labeled binding partner, comprise a standard which consists of at least one ADC3 peptide. A plurality of solutions with different, known ADC3 peptide concentrations are normally supplied as standard.

Obtaining of ADC3 Peptides and Anti-ADC3 Peptide Antibodies

A further embodiment of the invention is the obtaining of ADC3 peptides using recombinant expression systems, chromatographic methods and chemical synthesis protocols which are known to the skilled worker. The ADC3 peptides obtained in this way can be used inter alia as standards for quantifying the respective ADC3 peptides or as antigen for producing ADC3 peptide-specific antibodies. Methods known to the skilled worker and suitable for isolating and obtaining ADC3 peptides include the recombinant expression of peptides. It is possible to use for the expression of the ADC3 peptides inter alia cell systems such as, for example, bacteria such as Escherichia coli, yeast cells such as Saccharomyces cerevisiae, insect cells such as, for example, Spodoptera frugiperda (Sf-9) cells, or mammalian cells such as Chinese hamster ovary (CHO) cells. These cells are obtainable from the American Tissue Culture Collection (ATCC). For recombinant expression of ADC3 peptides, for example nucleic acid sequences which code for ADC3 peptides are inserted in combination with suitable regulatory nucleic acid sequences such as, for example, promoters, antibiotic selection markers etc. into an expression vector by molecular biology methods. A vector suitable for this purpose is, for example, the vector pcDNA3.1 from Invitrogen. The ADC3 peptide expression vectors obtained in this way can then be inserted into suitable cells, e.g. by electroporation. The ADC3 peptides can be prepared by chemical synthesis for example in accordance with the Merrifield solid-phase synthesis protocol using automatic synthesizers which are obtainable from various manufacturers. A further embodiment of this invention is the isolation of ADC3 peptides from biological samples or cell culture media or cell lysates from recombinant expression systems, e.g. using reverse phase chromatography, affinity chromatography, ion exchange chromatography, gel filtration, isoelectric focusing etc., or using other methods such as preparative immunoprecipitation, ammonium sulfate precipitation, extraction with organic solvents etc. A further embodiment of the invention is the obtaining of monoclonal or polyclonal antibodies using ADC3 peptides. The obtaining of antibodies takes place in the conventional way familiar to the skilled worker. A preferred embodiment of the production and obtaining of ADC3 peptide-specific antibodies which recognize neo-epitopes which are present only on ADC3 peptides but not in the complete complement C3 precursor molecule. Such anti-ADC3 peptide antibodies make the specific immunological detection of ADC3 peptides possible in the presence of the complement C3 precursor molecule.

Therapy Development and Monitoring Through ADC3 Peptide Determinations

A further exemplary use is the quantitative determination of the abovementioned ADC3 peptides for estimating the efficacy of a therapy under development for neurodegenerative diseases, in particular Alzheimer's disease. This entails comparison of quantitative measured results from a sample to be investigated with the measurements obtained in a control group and a group of patients. The efficacy of a therapeutic agent can be inferred from these results. The testing of efficacy is of outstanding importance for successful development of a therapeutic agent, and no clinically measurable parameter making this reliably possible is yet available for Alzheimer's disease [3].

Peptides which had undergone defined processing and defined post-translational modification and were derived from the amino acid sequence of the complement C3f molecule, and the C3f peptide itself, which are present in the cerebrospinal fluid from patients with Alzheimer's disease in a concentration which is specifically positively or negatively changed for each peptide, relative to the control group, were identified by using these methods.

Development of Therapeutic Applications of ADC3 Peptides

The concentrations of ADC3 peptides are changed markedly in patients with Alzheimer's disease compared with healthy people. A further aspect of the invention is therefore the bringing of the ADC3 concentrations to normal concentrations in patients with Alzheimer's disease. This method can be employed for the therapy of Alzheimer's disease or related neurological diseases. When complement C3 or ADC3 peptide concentrations are increased, the concentration of these substances can be reduced by therapeutic administration of, for example, anti-complement C3 or anti-ADC3 peptide antibodies or complement C3-specific antisense nucleic acids, ribozymes, RNAi nucleic acid molecules or triplex nucleic acids or ADC3 peptide antagonists or complement C3 antagonists. Substances which suppress the endogenous expression of complement C3 or the processing of complement C3 to ADC3 peptides can also be administered for therapy. If the disease is caused by a deficiency of complement C3 or ADC3 peptides, therapeutic doses of complement C3, ADC3 peptides, ADC3 peptide agonists or complement C3 agonists can be given. Substances which influence the processing of complement C3 to ADC3 peptides can also be employed in therapy. Combination of different therapeutic strategies is, of course, also possible and sensible in some circumstances.

The invention therefore also encompasses the use of complement C3 peptides, ADC3 peptides, ADC3 peptide agonists and ADC3 peptide antagonists, complement C3 peptide agonists and complement C3 peptide antagonists, anti-complement C3 antibodies and anti-ADC3 peptide antibodies for direct or indirect modulation of the concentration of complement C3 peptides and ADC3 peptides for the treatment of neurological diseases, in particular Alzheimer's disease. Alternative to antibodies, it is also possible to use antibody fragments, antibody fusion proteins or other substances which bind selectively to complement C3 peptides or ADC3 peptides. It is also possible as alternative to said proteins and peptides for fusion proteins of said proteins and peptides to be used. The invention further encompasses also the use of antisense nucleic acids, triplex nucleic acids, RNAi nucleic acid molecules, ribozymes and other nucleic acids which modulate the expression of said proteins and peptides. The invention additionally encompasses agonists and antagonists which modulate the activity of said proteins.

A further embodiment of the invention is the pharmaceutical formulation or chemical modification of the described peptides and nucleic acids to make it possible for them to cross the blood-brain barrier and/or the blood-CSF barrier more efficiently. They are thus made particularly suitable for therapeutic use. In order to achieve this, it is possible for example for ADC3 peptides, complement C3 peptides, peptidomimetics, nucleic acids, agonists or antagonists to be modified so that for example they become more lipophilic, favoring entry into the subarachnoid space, the cerebral ventricles and brain tissue. This can be achieved by introducing hydrophobic molecular constituents or else by “packaging” the substances in hydrophobic agents, e.g. liposomes. It is additionally possible for example for peptide sequences to be attached to ADC3 or complement C3 peptides, nucleic acids, agonists or antagonists, which favor entry into the subarachnoid space or, conversely, impede emergence from the subarachnoid space.

The invention also encompasses the administration of said therapeutic agents by various routes such as, for example, as intravenous injection, as substance which can be administered orally, as inhalable gas or aerosol, or administration in the form of direct injection into the subarachnoid space, the cerebral ventricles, or into tissue such as muscle, fat, brain etc. It is possible in this way to achieve increased bioavailability and efficacy, and an increased local concentration of these therapeutic agents. For example, peptides or proteins administered orally can be protected by acid-resistant capsules from proteolytic degradation in the stomach. Very hydrophobic substances can become more hydrophilic and thus better suited for, for example, intravenous injections by suitable pharmaceutical processing. Further possible dosage forms are inter alia packaging of the active ingredients in polymers and gels (Atrix Labs, Fort Collins, CO, USA, Andrx Pharmaceuticals, Davie, FL USA) etc.

A further embodiment of the invention is the use of ADC3 peptides or of complement C3 peptides for identifying receptors which selectively bind these molecules. These receptors can also be modulated by administration of agonists or antagonists, which is expedient for the therapy of neurological diseases, especially of Alzheimer's disease.

Exemplary embodiments of the examination of the therapeutic efficacy of complement C3 peptides, ADC3 peptides and of agents which modulate the expression and the bioavailability of these substances encompass the cultivation of cell lines. These cell lines can be treated with complement C3 peptides or ADC3 peptides or with peptidomimetics or with substances which promote the expression of complement C3 peptides or which promote the processing of complement C3 peptides to ADC3 peptides. It is possible thereby to establish the biological properties of complement C3 and ADC3 peptides in connection with neurological diseases, in particular Alzheimer's disease. Fusion proteins and fusion peptides can also be used for the treatment of the cell lines, e.g. fusion proteins with peptide sequences which promote transport of the fusion protein into the interior of the cell. Examples of possible fusion partners are HIV TAT, antennapedia, herpes simplex VP22 sequences, etc. It is likewise possible to transfect cell lines with expression vectors which bring about, directly or indirectly, expression of complement C3 peptides or ADC3 peptides by the transfected cells. These expression vectors may code inter alia for ADC3 peptides or complement C3 peptides. Simultaneous transfection with different ADC3 peptides and/or complement C3 peptides can also be carried out. Alternatively, suitable cell lines can be treated with anti-complement C3 peptide or anti-ADC3 peptide antibodies or with nucleic acids which suppress the expression of complement C3 peptides, such as, for example, complement C3 antisense nucleic acids, complement C3 triplex nucleic acids, complement C3 RNAi nucleic acids or ribozymes directed against complement C3 mRNA. Cell lines which appear suitable as neurological model systems in connection with complement C3 in particular can be used for such investigations. Read-out systems which can be used for these investigations are inter alia tests which measure the rate of proliferation of the treated cells, their metabolic activity, the rate of apoptosis of the cells, changes in cell morphology, in the expression of cell-intrinsic proteins or reporter genes or which measure the release of cytosolic cell constituents as markers for cell death.

Further test systems which can be used are suitable strains of experimental animals, e.g. of mice or rats or other species, which are considered as model of neurological diseases, in particular as model of Alzheimer's disease. These experimental animals can be used to investigate the efficacy of therapeutic strategies which aim to modulate the concentration of ADC3 peptides or of complement C3 peptides, it being possible for these peptides and proteins in some circumstances to be pharmaceutically processed so that they are better able to cross the blood-brain barrier and/or the blood-CSF barrier. It is possible to use as pharmaceutical processing method inter alia liposome-packaged proteins and peptides, proteins and peptides covalently fused or noncovalently associated with transport peptides such as, for example, the HIV TAT sequence etc. In addition, peptides and proteins can be chemically modified in such a way that they acquire lipophilic properties and are therefore able to penetrate more easily into cells. Peptides which are only slightly soluble in aqueous solutions can conversely be chemically modified so that they become more hydrophilic and then can be used for example as intravenously injectable therapeutic agent. Acid-resistant capsules can be used to protect sensitive substances, intended for oral administration, in the stomach.

Read-out parameters in experiments with animal models may be the survival time of the animals, their behaviour, their short-term memory, their learning ability, etc. One example of a memory test which is suitable for experimental animals such as, for example, rats is the Morris water maze test. Further parameters which can be used are the determination of body function such as, for example, blood tests, measurement of brain currents, metabolism tests, the expression of complement C3 peptides and ADC3 peptides and other proteins associated with the disease, and morphological and histological investigations on tissues such as, for example, the brain.

A further possibility for investigating the function of complement C3 peptides and ADC3 peptides is the possibility of obtaining experimental animals, by application of molecular biology methods, such as, for example, gene-deficient animals or transgenic animals in whose organism complement C3 peptides or ADC3 peptides are not produced, or are produced in reduced or increased amount. It is possible in this case for expression to be altered both locally, e.g. in the brain, and in the whole organism of the experimental animal. Suitable experimental animals are, inter alia, Caenorhabditis elegans, drosophila, zebra fish, mice, rats etc.

The invention is illustrated in detail below by means of examples. Reference is also made to the figures in this connection.

FIG. 1: Diagrammatic representation of the complement C3 protein with the position of the identified ADC3 peptides

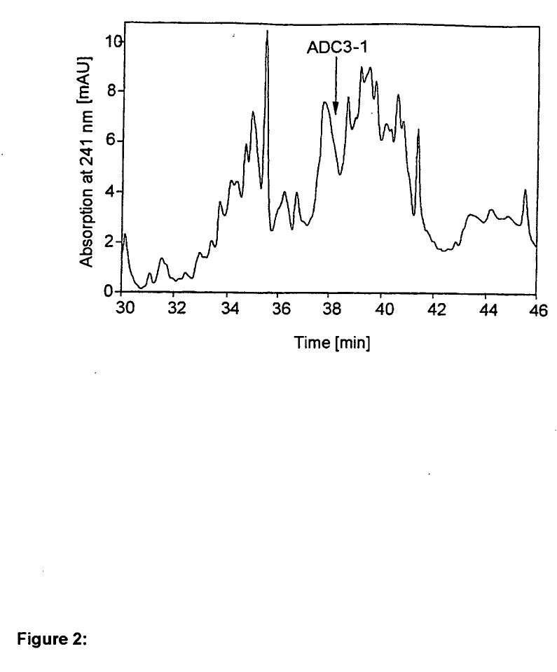

FIG. 2: Reverse phase chromatography for separation and concentration of the ADC3 peptides from cerebrospinal fluid

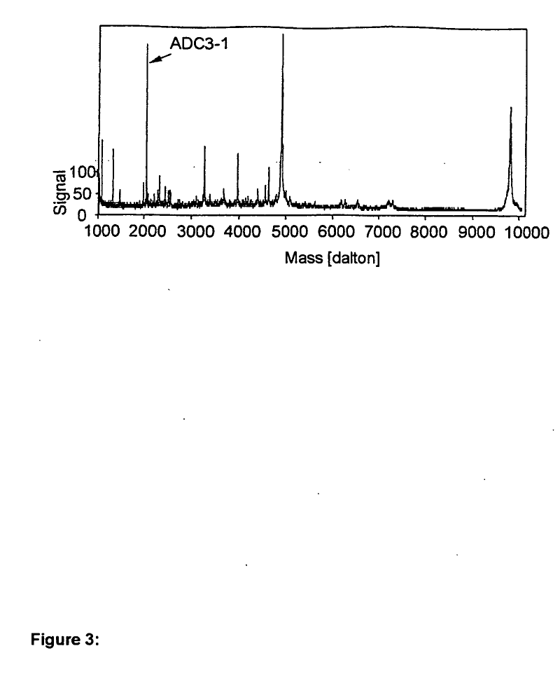

FIG. 3: Mass spectrometric measurement (MALDI) of the ADC3-1 peptide as example

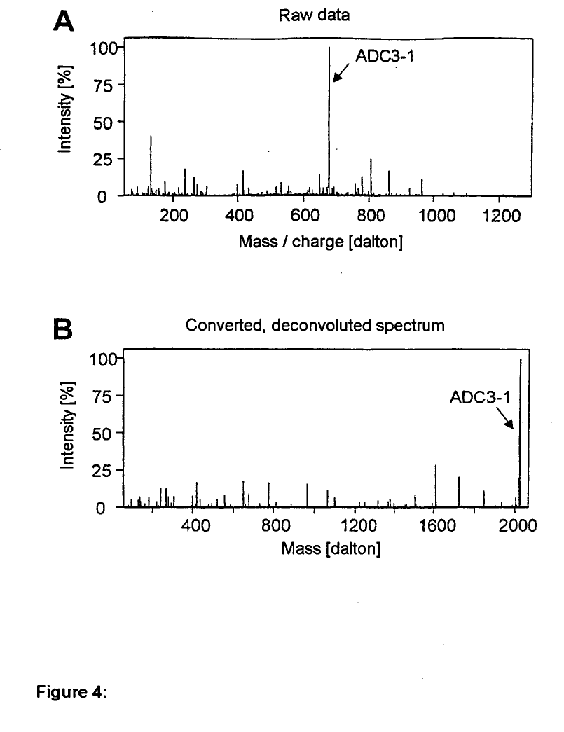

FIG. 4: MS/MS fragment spectrum for identifying peptides for ADC3-1 as example

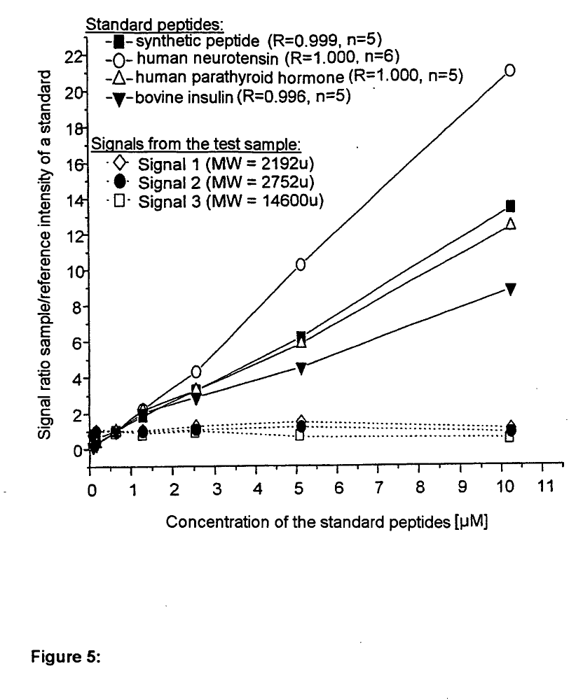

FIG. 5: MALDI as relatively quantifying mass spectroscopic method

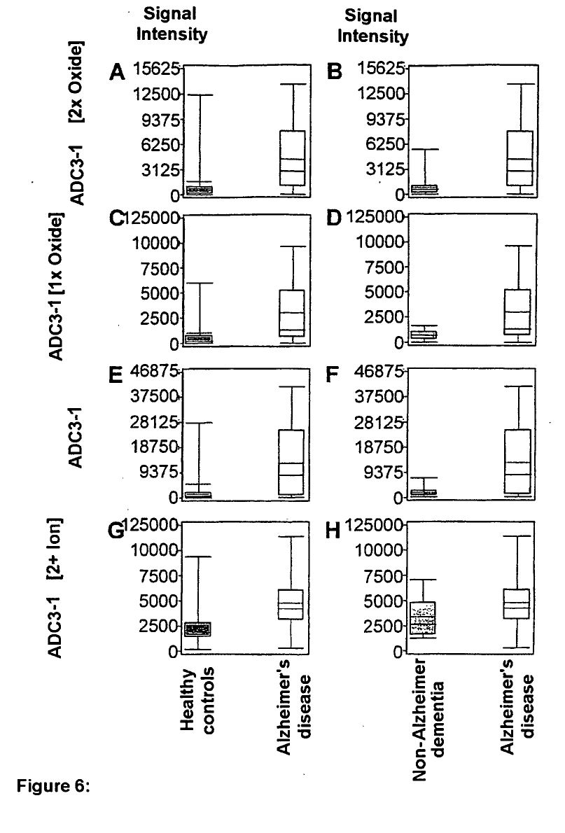

FIG. 6: Box-whisker plot for quantitative comparison of the concentrations of ADC3-1 in healthy controls, in patients with Alzheimer's disease and in patients with non-Alzheimer dementias, and of chemically modified ADC3-1 (e.g. the mono- or dioxidized peptide, and the doubly charged ADC3-1 peptide ion)

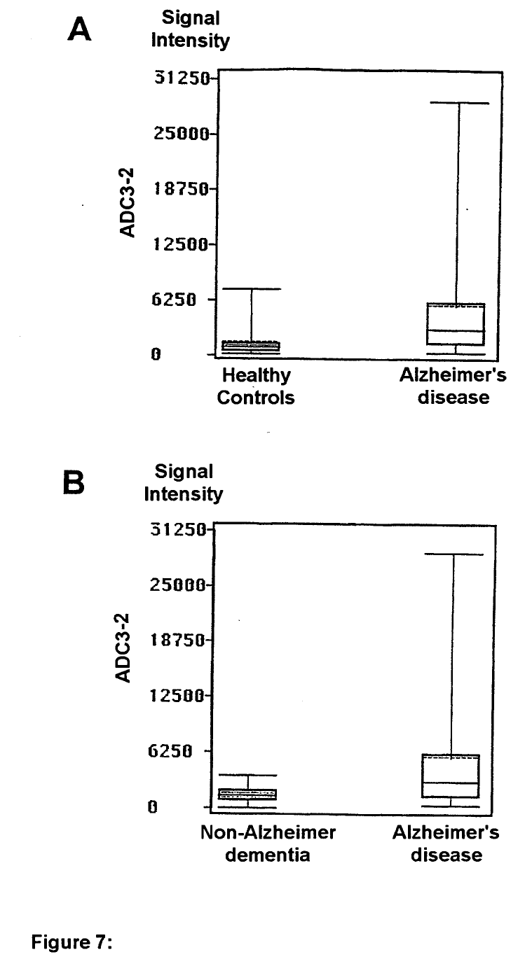

FIG. 7: Box-whisker plot for quantitative comparison of the concentrations of ADC3-2 in healthy controls, in patients with Alzheimer's disease and in patients with non-Alzheimer dementias

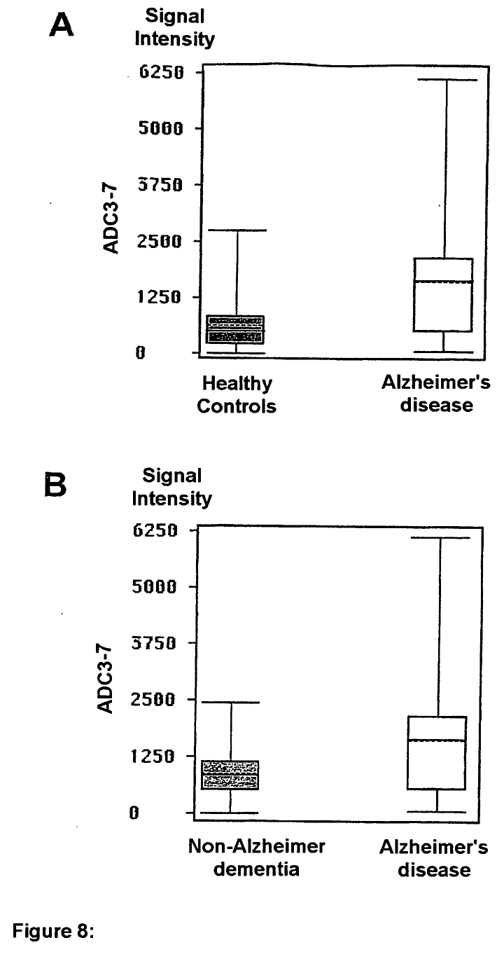

FIG. 8: Box-whisker plot for quantitative comparison of the concentrations of ADC3-7 in healthy controls, in patients with Alzheimer's disease and in patients with non-Alzheimer dementias

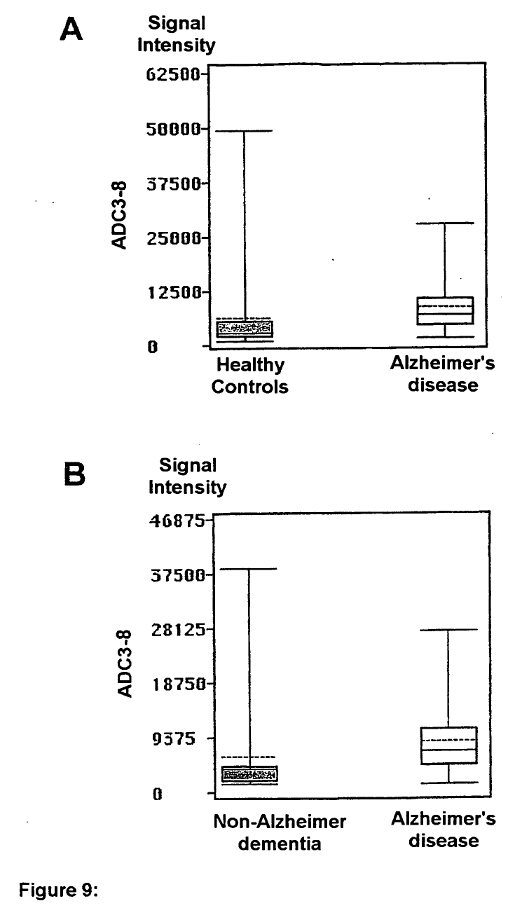

FIG. 9: Box-whisker plot for quantitative comparison of the concentrations of ADC3-8 in healthy controls, in patients with Alzheimer's disease and in patients with non-Alzheimer dementias

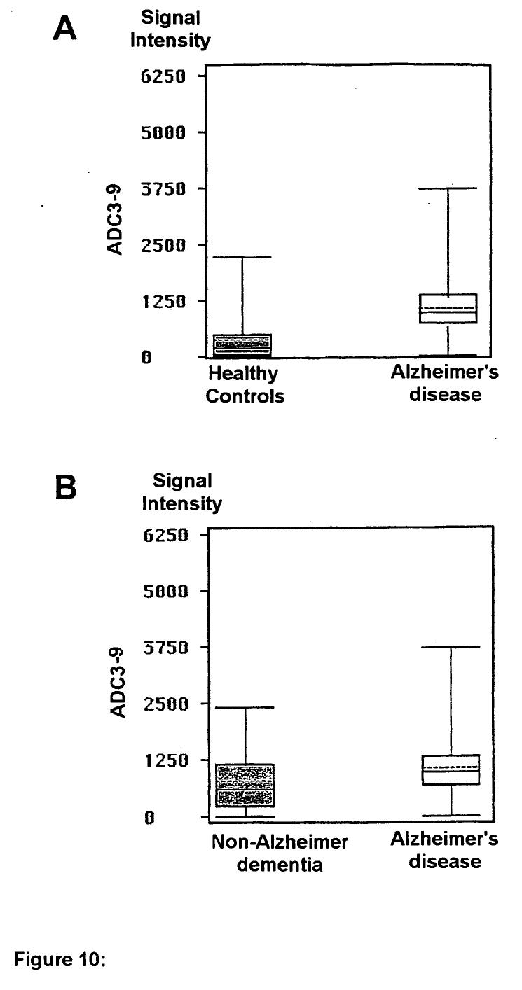

FIG. 10: Box-whisker plot for quantitative comparison of the concentrations of ADC3-9 in healthy controls, in patients with Alzheimer's disease and in patients with non-Alzheimer dementias

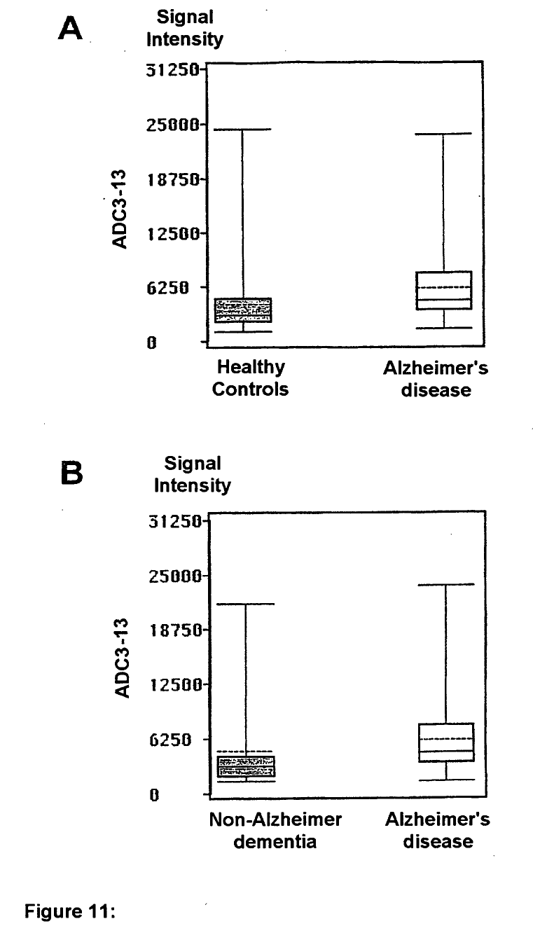

FIG. 11: Box-whisker plot for quantitative comparison of the concentrations of ADC3-13 in healthy controls, in patients with Alzheimer's disease and in patients with non-Alzheimer dementias

FIG. 1 shows a diagrammatic representation of the complement C3 peptide with the position of the identified ADC3 peptides which are depicted underneath each other in the form of an alignment. The theoretical monoisotopic masses of the peptides, stated in dalton, were calculated using the software GPMAW 4.02. They are: ADC3-1=2020.1/ADC3-2=1863.9/ADC3-3=1637.8/ADC3-4=1479.7/ADC3-5=1263.6/ADC3-6=1077.6/ADC3-7=1933.0/ADC3-8=1776.9/ADC3-9=1846.0/ADC3-10=1689.9/ADC3-11=1717.9/ADC3-12=1561.8/ADC3-13=1448.7/ADC3-14=1210.6/ADC3-15≧990.5 and ADC3-16≧941.4 dalton. The masses actually identified in the mass spectrometer differ from these theoretical monoisotopic masses because of the natural isotope distribution and of a small inaccuracy of measurement. The inaccuracy of measurement is 500 ppm for peptides in the mass range from 1000 to 4000 dalton, and the error of measurement for peptides with a mass greater than 4000 dalton increases slightly. Moreover, the measured mass of all the peptides is additionally increased by the mass of 1 proton (=1 dalton) owing to the method of measurement used. In addition, it was possible to identify the peptide ADC3-1 in the form of peptide variants having one and having two additional covalently linked oxygen atoms, the mass of the peptide being increased by about 16 dalton correspondingly for each oxygen atom. The masses experimentally determined for ADC3-1 in this connection are: 2038 and 2054 dalton, where the mass of ADC3-1 is increased in each case consecutively by the mass of one oxygen atom.

FIG. 2 shows the elution profile of a sample subjected to reverse phase chromatography as in Example 2 for separation and concentration of the ADC3 peptides from cerebrospinal fluid. The position at which the ADC3-1 peptides elute is marked by an arrow.

FIG. 3 shows a spectrum resulting from MALDI mass spectrometric measurement of ADC3-1 as in Example 3 after reverse phase chromatography of human cerebrospinal fluid as in Example 2. ADC3-1 corresponds to the sequence of the C3f peptide derived from complement C3 and is marked by an arrow.

FIG. 4 shows an MS/MS fragment spectrum as in Example 4 of the ADC3-1 peptide of the invention. FIG. 4A shows the raw data of the measurement, FIG. 4B shows the converted, deconvoluted mass spectrum of ADC₃₋₁. The peak pattern in FIG. 4B is characteristic of ADC₃₋₁. ADC3-1 corresponds to the C3f peptide of the complement C3 protein (Seq. ID 1).

FIG. 5 shows data generated by MALDI as relatively quantifying MS method. A sample was mixed with various amounts of different standard peptides, and the intensity both of these standard signals and of representative sample signals was measured. All signal intensities of the standards were standardized to their signal intensity at a concentration of 0.64 μM (=1). Each peptide shows an individual typical ratio of signal strength to concentration, which can be read off in this diagram from the gradient of the plot.

FIG. 6 shows box-whisker plots for the ADC3-1 peptide in its dioxidized form (FIGS. 6A and B), in its monooxidized form (FIGS. 6C and D), in its unoxidized form (FIGS. 6E and F) and as doubly charged ion (FIGS. 6G and H). The singly charged ion is analyzed in each of FIG. 6A to F. Each ADC3-1 variant is shown comparing patients with Alzheimer's disease and healthy controls (FIGS. 6A, C, E and G) and patients with Alzheimer's disease and non-Alzheimer patients (FIGS. 6B, D, F and H).

FIGS. 7 to 11 show in the form of box-whisker plots a comparison of the integrated MALDI mass spectroscopic signal intensities of ADC3-2, ADC3-7, ADC3-8, ADC3-9 and ADC3-13 in healthy controls compared with patients with Alzheimer's disease (Fig. A) and of non-Alzheimer dementias compared with patients with Alzheimer's disease (Fig. B).

### EXAMPLE 1

### Obtaining Cerebrospinal Fluid for Determining ADC3 Peptides

CSF or cerebrospinal fluid (fluid of the brain and spinal cord) is the fluid which is present in the four ventricles of the brain and in the subarachnoid space and which is produced in particular in the choroid plexus of the lateral ventricles. Cerebrospinal fluid is usually taken by lumbar puncture and less often by suboccipital puncture or ventricular puncture. In lumbar puncture (spinal puncture), to take cerebrospinal fluid, the puncture involves penetration of the spinal subarachnoid space between the 3rd and 4th or the 4th and 5th lumbar spinous process with a long hollow needle, and thus CSF being obtained. The sample is then centrifuged at 2000×g for 10 minutes, and the supernatant is stored at minus 70° C.

### EXAMPLE 2

### Separation of Peptides in Cerebrospinal Fluid (CSF) for Mass Spectrometric Measurement of ADC3 Peptides

For the detection of ADC3 peptides in CSF by mass spectrometry, it is necessary in this example to separate the peptide constituents. This sample pretreatment serves to concentrate the peptides of the invention and to remove components which may interfere with the measurement. The separation method carried out is a reverse phase chromatography. Various RP chromatography resins and eluents are equally suitable for this. The separation of ADC3 peptides using a C18 reverse phase chromatography column with the size of 4 mm×250 mm supplied by Vydac is described by way of example below. Mobile phases of the following composition were used: mobile phase A: 0.06% (v/v) trifluoroacetic acid, mobile phase B: 0.05% (v/v) trifluoroacetic acid, 80% (v/v) acetonitrile. Chromatography took place at 33° C. using an HP ChemStation 1100 supplied by Agilent Technologies with a micro flow cell supplied by Agilent Technologies. Human cerebrospinal fluid was used as sample. 440 μl of CSF were diluted with water to 1650 μl, the pH was adjusted to 2-3, the sample was centrifuged at 18 000×g for 10 minutes and finally 1500 μl of the sample prepared in this way were loaded onto the chromatography column. The chromatography conditions were as follows: 5% mobile phase B at time 0 min, from time 1 to 45 min continuous increase in the mobile phase B concentration to 50%, from time 45 to 49 min continuous increase in the mobile phase B concentration to 100% and subsequently up to time 53 min constant 100% buffer B. Collection of 96 fractions each of 0.5 ml starts 10 minutes after the start of the chromatography. The chromatogram of a cerebrospinal fluid sample prepared under the experimental conditions described herein is depicted in FIG. 2.

### EXAMPLE 3

### Measurement of Masses of Peptides by Means of MALDI Mass Spectrometry

For mass analysis, typical positive ion spectra of peptides are produced in a MALDI-TOF mass spectrometer (matrix-assisted laser desorption ionization). Suitable MALDI-TOF mass spectrometers are manufactured by PerSeptive Biosystems Framingham (Voyager-DE, Voyager-DE PRO or Voyager-DE STR) or by Bruker Daltonik Bremen (BIFLEX). The samples are prepared by mixing them with a matrix substance which typically consists of an organic acid. Typical matrix substances suitable for peptides are 3,5-dimethoxy-4-hydroxycinnamic acid, α-cyano-4-hydroxycinnamic acid and 2,5-dihydroxybenzoic acid. A dried equivalent obtained by reverse phase chromatography and corresponding to 400 μl of human cerebrospinal fluid is used to measure the ADC3 peptides of the invention. The chromatographed sample is dissolved in 15 μl of a matrix solution. This matrix solution contains, for example, 10 g/l α-cyano-4-hydroxycinnamic acid and 10 g/l L(−)fucose dissolved in a solvent mixture consisting of acetonitrile, water, trifluoroacetic acid and acetone in the ratio 49:49:1:1 by volume. 0.3 μl of this solution is transferred to a MALDI carrier plate, and the dried sample is analyzed in a Voyager-DE STR MALDI mass spectrometer from PerSeptive Biosystems. The measurement takes place in linear mode with delayed extraction™. As example of a measurement of one of the ADC3 peptides of the invention, FIG. 3 shows the spectrum of ADC3-1. The peak corresponding to the ADC3-1 peptide is marked by an arrow.

The MALDI-TOF mass spectrometry can be employed to quantify peptides such as, for example, the ADC3 peptides of the invention if these peptides are present in a concentration which is within the dynamic measurement range of the mass spectrometer, thus avoiding detector saturation. This is the case for the measurement of the ADC3 peptides of the invention in cerebrospinal fluid at a CSF equivalent concentration of 33.3 μl per μl of matrix solution. There is a specific ratio between measured signal and concentration for each peptide, which means that the MALDI mass spectrometry can preferably be used for the relative quantification of peptides. This situation is depicted in FIG. 5. If various amounts of different standard peptides are added to a sample, it is possible to measure the intensity both of these standard signals and of the sample signals. FIG. 5 shows by way of example a MALDI measurement as relatively quantifying MS method. All signal intensities of the standards were standardized to their signal intensity at a concentration of 0.64 μmol (=1). Each peptide shows an individual, typical ratio of signal strength to concentration, which can be read off from the gradient of the plot.

### EXAMPLE 4

### Mass Spectrometric Identification of the ADC3 Peptides

For quantification of the ADC3 peptides of the invention it is necessary to ensure that the mass signals to be analyzed of peptides in the fractions obtained by reverse phase chromatography of cerebrospinal fluid, as in Example 2, in fact relate to the ADC3 peptides of the invention. The peptides of the invention in these fractions are identified for example using nanoSpray-MS/MS [11]. This entails an ADC3 peptide ion in the mass spectrometer being selected in the mass spectrometer on the basis of its specific m/z (mass/charge) value in a manner known to the skilled worker. This selected ion is then fragmented by supplying collisional energy with an impinging gas, e.g. argon or nitrogen, and the resulting ADC3 peptide fragments are detected in the mass spectrometer in an integrated analysis unit, and corresponding m/z values are determined (principle of tandem mass spectrometry) [15]. The fragmentation behavior of peptides makes unambiguous identification of the ADC3 peptides of the invention possible when the accuracy of mass of the instruments used is, for example, 50 ppm by the use of computer-assisted search methods [16] in sequence databases into which the sequence of the complement C3 precursor molecule has been entered. Oxidized peptides can also be identified by this method, taking into account the positive shift of the molecular mass by about 16 dalton, corresponding to the mass of one oxygen atom. Other chemically or post-translationally modified peptides can also be identified correspondingly. In this specific case, the mass spectrometric analysis took place with a quadrupole TOF Instrument, QStar-Pulsar model from Applied Biosystems-Sciex, USA. Examples of MS/MS fragment spectra are shown in FIG. 4.

### EXAMPLE 5

### Mass Spectrometric Quantification of ADC3 Peptides to Compare their Relative Concentration in Control and Patients' Samples

A sample preparation as in Example 1 and 2 followed by a MALDI measurement of the ADC3 peptides of the invention as in Example 3 were carried out for control samples and for samples from patients suffering from Alzheimer's disease. Examples of MALDI signal intensities are depicted in the form of box-whisker plots in FIGS. 6 to 11. The box-whisker plots depicted make it possible to compare the integrated MALDI mass spectrometric signal intensities of various ADC3 peptides in controls (healthy subjects, patients with non-Alzheimer dementias) with the MALDI signal intensities in samples from Alzheimer's disease patients. In these, the box, i.e. the columns in the diagrams in FIGS. 6 to 11, in each case includes the range in which 50% of the respective MALDI signal intensities are to be found (2nd and 3rd quartile), and the lines starting from the box and pointing upward and downward (whiskers) indicate the range in which in each case the 25% of measurements which show the highest signal intensities (upper quartile) are to be found, and in which the 25% of measurements which show the lowest signal intensities (lower quartile) are to be found. The full line in the columns indicates the median and the broken line in the columns indicates the mean.

### REFERENCES

1. Beers, M. H., and R. Berkow, 1999. Delirium and dementia. In The Merck Manual of Diagnosis and Therapy, 17th Ed. Merck Research Laboratories, Whitehouse Station, NJ, USA. Pages 1389-1400.
2. Beers, M. H., and R. Berkow, 1999. Neurological approach to the patient. In The Merck Manual of Diagnosis and Therapy, 17th Ed. Merck Research Laboratories, Whitehouse Station, NJ, USA. Page 1345.
3. Engelborghs, S., and P. P. De Deyn. 2001. Biological and genetic markers of sporadic Alzheimer's disease. Acta Med Okayama. 55:55-63.
4. Harrison, R. A., T. C. Ferries, F. D. Northrop, P. J. Lachmann, and A. E. Davis. 1988. Structure of C3f, a small peptide specifically released during inactivation of the third component of complement. Complement. 5:27-32.
5. Devereux, J., P. Haeberli, and O. Smithies. 1984. A comprehensive set of sequence analysis programs for the VAX. Nucleic Acids Res. 12:387-95.

6. Altschul, S. F., W. Gish, W. Miller, E. W. Myers, and D. J. Lipman. 1990. Basic local alignment search tool. J Mol. Biol. 215:403-10.
7. Needleman, S. B., and C. D. Wunsch. 1970. A general method applicable to the search for similarities in the amino acid sequences of two proteins. J Mol. Biol. 48:443-53.

8. Henikoff, S., and J. G. Henikoff. 1992. Amino acid substitution matrices from protein blocks. Proc Natl Acad Sci USA. 89:10915-9.
9. Garavelli, J. S., Z. Hou, N. Pattabiraman, and R. M. Stephens. 2001. The RESID Database of protein structure modifications and the NRL-3D Sequence-Structure Database. Nucleic Acids Res. 29:199-201.
10. Beers, M. H., and R. Berkow, 1999. Biology of the immune system. In The Merck Manual of Diagnosis and Therapy, 17th Ed. Merck Research Laboratories, Whitehouse Station, NJ, USA. Pages 1002-1023.
11. Ganu, V. S., H. J. Muller-Eberhard, and T. E. Hugli. 1989. Factor C3f is a spasmodic fragment released from C3b by factors I and H: the heptadeca-peptide C3f was synthesized and characterized. Mol Immunol. 26:939-48.
12. Yasojima, K., C. Schwab, E. G. McGeer, and P. L. McGeer. 1999. Up-regulated production and activation of the complement system in Alzheimer's disease brain. Am J Pathol. 154:927-36.
13. Eikelenboom, P., and F. C. Stam. 1982. Immunoglobulins and complement factors in senile plaques. An immunoperoxidase study. Acta Neuropathol. 57:239-42.

1. A method for detecting Alzheimer's disease or a predisposition to Alzheimer's disease through determination of at least one ADC3 marker peptide or a peptide which is derived from the sequence having the Swiss prot accession no. P01024 or a sequence homologous thereto in an individual's biological sample.

2. A method for detecting Alzheimer's disease or a predisposition to Alzheimer's disease with determination of the concentration of at least one marker peptide in a patient's biological sample, characterized in that
a) at least one ADC3 peptide or a marker peptide derived from the sequence having the Swiss prot accession no. P01024 or a sequence homologous thereto is used as marker peptide,
b) a concentration increase or concentration reduction which is specific for the particular marker peptide is found in the sample,
c) a significant marker peptide concentration change in the manner mentioned under b) is regarded as positive detection result for Alzheimer's disease.

3. The method as claimed in claim 1, characterized in that the peptide is present in the sample in post-translational modifications or in chemically modified form, preferably as peptide oxide, and is detected in this form.

4. The method as claimed in claim 1, characterized in that the biological sample is cerebrospinal fluid, serum, plasma, urine, synovial fluid, stool, tear fluid, lymph or a tissue sample or cell sample or is obtained therefrom by processing.

5. The method as claimed in claim 1, characterized in that the ADC3 peptides are determined with the aid of a mass spectrometric or of a biological activity assay or of a molecular biology or of an immunological assay, preferably with the aid of ADC3 peptide-binding phage particles, PNAs, antibodies, affinity matrices or of an ELISA assay.

6. The method as claimed in claim 1 characterized in that before the determination the sample is fractionated by chromatography and/or subjected to a precipitation reaction and/or a liquid phase separation, with the resulting fractions subsequently being investigated separately.

7. The method as claimed in claim 1, characterized
a) in that the method is carried out to increase the sensitivity and/or specificity of the diagnosis in combination with other diagnostic methods or
b) in that the concentration of the marker peptide is used as measure of the severity of the disease.

8. A method for obtaining an ADC3 peptide, including pos-translationally or chemically modified peptides by isolation from a biological sample, by recombinant production or by chemical synthesis, where the ADC3 peptides
a) are peptides as shown in Seq. ID 1 to Seq. ID 16, or
b) are derivatives of naturally occurring complement C3 alleles of these peptides, or
c) are ADC3 mutants preferably having not more than 2 different amino acids compared with the corresponding section of the complement C3 sequence.

9. Peptides as shown in Seq. ID 3 to Seq. ID 16, or peptides which are homologous to peptides corresponding to Seq. ID 1 to Seg. ID 16, in particular derivatives of naturally occurring complement C3 alleles or point-mutated, or post-translationally, enzymatically or chemically modified, preferably oxidized ADC3 peptides, or peptidomimetics corresponding to the aforementioned petides.

10. The use of at least one of the peptides as claimed in claim 9 or of peptides which are homologous to the peptide having the Swiss prot accession no. P01024 for the production or development of a diagnostic reagent or of a therapeutic agent, in particular for obtaining antibodies as diagnostic reagent for Alzheimer's disease.

11. Antibodies which are directed against ADC3 peptides.

12. The use of nucleic acids corresponding to the peptides as shown in Seq. ID 1 to Seq. ID 16 or to the sequence having the NCBI accession no. XM_{—}009010 for indirect determination of the relative concentration of the relevant peptides and peptide fragments for diagnosing Alzheimer's disease.

13. Nucleic acids which code for ADC3 peptides, or nucleic acids which are complementary to nucleic acids which code for ADC3 peptides.

14. Medicaments or diagnostic aids comprising ADC3 peptides or corresponding peptidomimetics or comprising antibodies against ADC3 peptides or comprising nucleic acids which code for ADC3 peptides or nucleic acids which are complementary to nucleic acids which code for ADC3 peptides.

15. Medicaments or diagnostic aids comprising substances corresponding to claim 14 and at least one further pharmacologically acceptable substance, preferably a solvent, a filler, a preservative or a color or a flavoring.

16. A test kit for detecting Alzheimer's disease or a predisposition to Alzheimer's disease comprising at least one antibody which is directed against an ADC3 peptide and which is present in immobilized or labeled form, or in a form which makes immobilization or labeling possible.

17. The test kit as claimed in claim 16, characterized in that a standard consisting of at least one ADC3 peptide is additionally present.

18. The method as claimed in claim 2, characterized in that the peptide is present in the sample in post-translational modifications or in chemically modified form, preferably as peptide oxide, and is detected in this form.

19. The method as claimed in claim 2, characterized in that the biological sample is cerebrospinal fluid, serum, plasma, urine, synovial fluid, stool, tear fluid, lymph or a tissue sample or cell sample or is obtained therefrom by processing.

20. The method as claimed in claim 2, characterized in that the ADC3 peptides are determined with the aid of a mass spectrometric or of a biological activity assay or of a molecular biology or of an immunological assay, preferably with the aid of ADC3 peptide-binding phage particles, PNAs, antibodies, affinity matrices or of an ELISA assay.

21. The method as claimed in claim 2, characterized in that before the determination he sample is fractionated by chromatography and/or subjected to a precipitation reaction and/or a liquid phase separation, with the resulting fractions subsequently being investigated separately.

22. The method as claimed in claim 2, characterized
a) in that the method is carried out to increase the sensitivity and/or specificity of the diagnosis in combination with other diagnostic methods or
b) in that the concentration of the marker peptide is used as measure of the severity of the disease.


SEQUENCE LISTING (18 sequences)

SEQ ID NO: 1 - protein, 17 aa, homo sapiens
SSKITHRIHWESASLLR

SEQ ID NO: 2 - protein, 16 aa, homo sapiens
SSKITHRIHWESASLL

SEQ ID NO: 3 - protein, 14 aa, homo sapiens
SSKITHRIHWESAS

SEQ ID NO: 4 - protein, 12 aa, homo sapiens
SSKITHRIHWES

SEQ ID NO: 5 - protein, 10 aa, homo sapiens
SSKITHRIHW

SEQ ID NO: 6 - protein, 9 aa, homo sapiens
SSKITHRIH

SEQ ID NO: 7 - protein, 16 aa, homo sapiens
SKITHRIHWESASLLR

SEQ ID NO: 8 - protein, 15 aa, homo sapiens
SKITHRIHWESASLL

SEQ ID NO: 9 - protein, 15 aa, homo sapiens
KITHRIHWESASLLR

SEQ ID NO: 10 - protein, 14 aa, homo sapiens
KITHRIHWESASLL

SEQ ID NO: 11 - protein, 14 aa, homo sapiens
ITHRIHWESASLLR

SEQ ID NO: 12 - protein, 13 aa, homo sapiens
ITHRIHWESASLL

SEQ ID NO: 13 - protein, 12 aa, homo sapiens
THRIHWESASLL

SEQ ID NO: 14 - protein, 10 aa, homo sapiens
IHWESASLLR

SEQ ID NO: 15 - protein, 8 aa, homo sapiens
  MISC_FEATURE: N-terminal extension r1 possible; r1 represents the amino acid Ser or no amino acid
SKITHRIH

SEQ ID NO: 16 - protein, 8 aa, homo sapiens
  MISC_FEATURE: C-terminal extension r4 possible; r4 represents the amino acid Arg or no amino acid
HWESASLL

SEQ ID NO: 17 - protein, 1255 aa, Homo sapiens
MGPTSGPSLLLLLLTHLPLALGSPMYSIITPNILRLESEETMVLEAHDAQGDVPVTVTVHDFPGKKLVLSSEKTVLTPATNHMGNVTFTIPANREFKSEKGRNKFVTVQATFGTQVVEKVVLVSLQSGYLFIQTDKTIYTPGSTVLYRIFTVNHKLLPVGRTVMVNIENPEGIPVKQDSLSSQNQLGVLPLSWDIPELVNMGQWKIRAYYENSPQQVFSTEFEVKEYVLPSFEVIVEPTEKFYYIYNEKGLEVTITARFLYGKKVEGTAFVIFGIQDGEQRISLPESLKRIPIEDGSGEVVLSRKVLLDGVQNLRAEDLVGKSLYVSATVILHSGSDMVQAERSGIPIVTSPYQIHFTKTPKYFKPGMPFDLMVFVTNPDGSPAYRVPVAVQGEDTVQSLTQGDGVAKLSINTHPSQKPLSITVRTKKQELSEAEQATRTMQALPYSTVGNSNNYLHLSVLRTELRPGETLNVNFLLRMDRAHEAKIRYYTYLIMNKGRLLKAGRQVREPGQDLVVLPLSITTDFIPSFRLVAYYTLIGASGQREVVADSVWVDVKDSCVGSLVVKSGQSEDRQPVPGQQMTLKIEGDHGARVVLVAVDKGVFVLNKKNKLTQSKIWDVVEKADIGCTPGSGKDYAGVFSDAGLTFQQLAFRQPSSAFAAFVKRAPSTWLTAYVVKVFSLAVNLIAIDSQVLCGAVKWLILEKQKPDGVFQEDAPVIHQEMIGGLRNNNEKDMALTAFVLISLQEAKDICEEQVNSLPGSITKAGDFLEANYMNLQRSYTVAIAGYALAQMGRLKGPLLNKFLTTAKDKNRWEDPGKQLYNVEATSYALLALLQLKDFDFVPPVVRWLNEQRYYGGGYGSTQATFMVFQALAQYQKDAPDHQELNLDVSLQLPSRSSKITHRIHWESASLLRSEETKENEGFTVTAEGKGQGTLSVVTMYHAKAKDQLTCNKFDLKVTIKPAPETEKRPQDAKNTMILEICTRYRGDQDATMSILDISMMTGFAPDTDDLKQLANGVDRYISKYELDKAFSDRNTLIIYLDKVSHSEDDCLAFKVHQYFNVELIQPGAVKVYAYYNLEESCTRFYHPEKEDGKLNKLCRDELCRCAEENCFIQKSDDKVTLEERLDKACEPGVDYVYKTRLVKVQLSNDFDEYIMAIEQTIKSGSDEVQVGQQRTFISPIKCREALKLEEKKHYLMWGLSSDFWGEKPNLSYIIGKDTWVEHWPEEDECQDEENQKQCQDLGAFTESMVVFGCPN

SEQ ID NO: 18 - DNA, 5067 bp, Homo sapiens
ctcctccccatcctctccctctgtccctctgtccctctgaccctgcactgtcccagcaccatgggacccacctcaggtcccagcctgctgctcctgctactaacccacctccccctggctctggggagtcccatgtactctatcatcacccccaacatcttgcggctggagagcgaggagaccatggtgctggaggcccacgacgcgcaaggggatgttccagtcactgttactgtccacgacttcccaggcaaaaaactagtgctgtccagtgagaagactgtgctgacccctgccaccaaccacatgggcaacgtcaccttcacgatcccagccaacagggagttcaagtcagaaaaggggcgcaacaagttcgtgaccgtgcaggccaccttcgggacccaagtggtggagaaggtggtgctggtcagcctgcagagcgggtacctcttcatccagacagacaagaccatctacacccctggctccacagttctctatcggatcttcaccgtcaaccacaagctgctacccgtgggccggacggtcatggtcaacattgagaacccggaaggcatcccggtcaagcaggactccttgtcttctcagaaccagcttggcgtcttgcccttgtcttgggacattccggaactcgtcaacatgggccagtggaagatccgagcctactatgaaaactcaccacagcaggtcttctccactgagtttgaggtgaaggagtacgtgctgcccagtttcgaggtcatagtggagcctacagagaaattctactacatctataacgagaagggcctggaggtcaccatcaccgccaggttcctctacgggaagaaagtggagggaactgcctttgtcatcttcgggatccaggatggcgaacagaggatttccctgcctgaatccctcaagcgcattccgattgaggatggctcgggggaggttgtgctgagccggaaggtactgctggacggggtgcagaacccccgagcagaagacctggtggggaagtctttgtacgtgtctgccaccgtcatcttgcactcaggcagtgacatggtgcaggcagagcgcagcgggatccccatcgtgacctctccctaccagatccacttcaccaagacacccaagtacttcaaaccaggaatgccctttgacctcatggtgttcgtgacgaaccctgatggctctccagcctaccgagtccccgtggcagtccagggcgaggacactgtgcagtctctaacccagggagatggcgtggccaaactcagcatcaacacacaccccagccagaagcccttgagcatcacggtgcgcacgaagaagcaggagctctcggaggcagagcaggctaccaggaccatgcaggctctgccctacagcaccgtgggcaactccaacaattacctgcatctctcagtgctacgtacagagctcagacccggggagaccctcaacgtcaacttcctcctgcgaatggaccgcgcccacgaggccaagatccgctactacacctacctgatcatgaacaagggcaggctgttgaaggcgggacgccaggtgcgagagcccggccaggacctggtggtgctgcccctgtccatcaccaccgacttcatcccttccttccgcctggtggcgtactacacgctgatcggtgccagcggccagagggaggtggtggccgactccgtgtgggtggacgtcaaggactcctgcgtgggctcgctggtggtaaaaagcggccagtcagaagaccggcagcctgtacctgggcagcagatgaccctgaagatagagggtgaccacggggcccgggtggtactggtggccgtggacaagggcgtgttcgtgctgaataagaagaacaaactgacgcagagtaagatctgggacgtggtggagaaggcagacatcggctgcaccccgggcagtgggaaggattacgccggtgtcttctccgacgcagggctgaccttcacgagcagcagtggccagcagaccgcccagagggcagaacttcagtgcccgcagccagccgcccgccgacgccgttccgtgcagctcacggagaagcgaatggacaaagtcggcaagtaccccaaggagctgcgcaagtgctgcgaggacggcatgcgggagaaccccatgaggttctcgtgccagcgccggacccgtttcatctccctgggcgaggcgtgcaagaaggtcttcctggactgctgcaactacatcacagagctgcggcggcagcacgcgcgggccagccacctgggcctggccaggagtaacctggatgaggacatcattgcagaagagaacatcgtttcccgaagtgagttcccagagagctggctgtggaacgttgaggacttgaaagagccaccgaaaaatggaatctctacgaagctcatgaatatatttttgaaagactccatcaccacgtgggagattctggctgtgagcatgtcggacaagaaagggatctgtgtggcagaccccttcgaggtcacagtaatgcaggacttcttcatcgacctgcggctaccctactctgttgttcgaaacgagcaggtggaaatccgagccgttctctacaattaccggcagaaccaagagctcaaggtgagggtggaactactccacaatccagccttctgcagcctggccaccaccaagaggcgtcaccagcagaccgtaaccatcccccccaagtcctcgttgtccgttccatatgtcatcgtgccgctaaagaccggcctgcaggaagtggaagtcaaggctgctgtctaccatcatttcatcagtgacggtgtcaggaagtccctgaaggtcgtgccggaaggaatcagaatgaacaaaactgtggctgttcgcaccctggatccagaacgcctgggccgtgaaggagtgcagaaagaggacatcccacctgcagacctcagtgaccaagtcccggacaccgagtctgagaccagaattctcctgcaagggaccccagtggcccagatgacagaggatgccgtcgacgcggaacggctgaagcacctcattgtgaccccctcgggctgcggggaacagaacatgatcggcatgacgcccacggtcatcgctgtgcattacctggatgaaacggagcagtgggagaagttcggcctagagaagcggcagggggccttggagctcatcaagaaggggtacacccagcagctggccttcagacaacccagctctgcctttgcggccttcgtgaaacgggcacccagcacctggctgaccgcctacgtggtcaaggtcttctctctggctgtcaacctcatcgccatcgactcccaagtcctctgcggggctgttaaatggctgatcctggagaagcagaagcccgacggggtcttccaggaggatgcgcccgtgatacaccaagaaatgattggtggattacggaacaacaacgagaaagacatggccctcacggcctttgttctcatctcgctgcaggaggctaaagatatttgcgaggagcaggtcaacagcctgccaggcagcatcactaaagcaggagacttccttgaagccaactacatgaacctacagagatcctacactgtggccattgctggctatgctctggcccagatgggcaggctgaaggggcctcttcttaacaaatttctgaccacagccaaagataagaaccgctgggaggaccctggtaagcagctctacaacgtggaggccacatcctatgccctcttggccctactgcagctaaaagactttgactttgtgcctcccgtcgtgcgttggctcaatgaacagagatactacggtggtggctatggctctacccaggccaccttcatggtgttccaagccttggctcaataccaaaaggacgcccctgaccaccaggaactgaaccttgatgtgtccctccaactgcccagccgcagctccaagatcacccaccgtatccactgggaatctgccagcctcctgcgatcagaagagaccaaggaaaatgagggtttcacagtcacagctgaaggaaaaggccaaggcaccttgtcggtggtgacaatgtaccatgctaaggccaaagatcaactcacctgtaataaattcgacctcaaggtcaccataaaaccagcaccggaaacagaaaagaggcctcaggatgccaagaacactatgatccttgagatctgtaccaggtaccggggagaccaggatgccactatgtctatattggacatatccatgatgactggctttgctccagacacagatgacctgaagcagctggccaatggtgttgacagatacatctccaagtatgagctggacaaagccttctccgataggaacaccctcatcatctacctggacaaggtctcacactctgaggatgactgtctagctttcaaagttcaccaatactttaatgtagagcttatccagcctggagcagtcaaggtctacgcctattacaacctggaggaaagctgtacccggttctaccatccggaaaaggaggatggaaagctgaacaagctctgccgtgatgaactgtgccgctgtgctgaggagaattgcttcatacaaaagtcggatgacaaggtcaccctggaagaacggctggacaaggcctgtgagccaggagtggactatgtgtacaagacccgactggtcaaggttcagctgtccaatgactttgacgagtacatcatggccattgagcagaccatcaagtcaggctcggatgaggtgcaggttggacagcagcgcacgttcatcagccccatcaagtgcagagaagccctgaagctggaggagaagaaacactacctcatgtggggtctctcctccgatttctggggagagaagcccaacctcagctacatcatcgggaaggacacttgggtggagcactggcccgaggaggacgaatgccaagacgaagagaaccagaaacaatgccaggacctcggcgccttcaccgagagcatggttgtctttgggtgccccaactgaccacacccccattcc